(19) United States
(12) Patent Application Publication
(10) Pub. No.: US 2018/0289944 A1
(43) Pub. Date: Oct. 11, 2018

(54) Aseptic or sterile connection system

(71) Applicant: Warner B. Roberts, Copley, OH (US)

(72) Inventor: Warner B. Roberts, Copley, OH (US)

(21) Appl. No.: 15/941,922

(22) Filed: Mar. 30, 2018

Related U.S. Application Data
(60) Provisional application No. 62/483,981, filed on Apr. 11, 2017.

(51) Int. Cl. A61M 39/16; A61M 39/24; A61M 39/18

(52) U.S. Cl. CPC A61M 39/162; A61M 39/1011; A61M 39/18; A61M 39/24

(57) ABSTRACT

A means and method that forms and maintains an enclosed sterile environment that then is able to sterilize all that is within the enclosure in order to facilitate making a sterile or aseptic connection and keeping it sterile or aseptic. Connection may be between a container of material to a and through a terminus using tubes or hoses interspersed with a connector or connectors. The means and method are substantially comprised of an enclosure to be filled with a sterilant, that by various means attaches to or are parts of connectors, other connectors, a sterilant, a method of propelling the sterilant and other propelling mechanisms as required. The sterilant surrounds the critical parts to make all that is within an enclosure including the connectors and other parts within the enclosure as well as possibly through hoses or tubes to a terminus and through it to the point a material exits the terminus while making a connection sterile or aseptic. It is also the means of repeating the process of connecting a source to a terminus sterile or aseptic. Other aspects may also include maintaining sterility of other points of connection.

### CROSS-REFERENCE TO RELATED APPLICATIONS

This application claims priority to U.S. Provisional Patent application No. 62/483,981 which was filed on Apr. 11, 2017

### STATEMENT REGARDING FEDERALLY SPONSORED RESEARCH OR DEVELOPMENT

NA

### REFERENCE TO A “MICROFICHE APPENDIX”

NA

### BACKGROUND OF THE INVENTION

### (1) Field of the Invention

A means and method to facilitate connecting, more specifically a means and method to facilitate the connecting of tubes or hoses that may be interspersed by and attached to connectors. They may be from an aseptic material container with a material connector attached to a terminus connector, that may have tubes or hoses attached, that may then lead to a terminus. The connectors may be within or critical elements may be within an enclosure that creates an environment in which to make the process and completion of connecting the connectors sterile or aseptic.

### (2) Description of the Related Art

A continuing need exists to prevent contamination of a material, such as a fluid, where a material is needed to be transitioned from a container to a terminus in an uncontaminated condition. The means and method of making connections of hoses or tubes could be comprised of interspersed connectors that lead from a material container through hoses or tubes, that may have additional connectors and through to other hoses or tubes and other connectors that then lead to a terminus. Its use would be applicable in a multitude of industries among them medical, bio-medical, food and beverage.

It is the object of the invention to improve upon systems that allow for making an aseptic connection so that all parts of a system that may need to be, are sterilized to be made sterile. It draws upon asepsis concepts used in isolating procedures such as in glove boxes, clean rooms, aseptic fillers and others to sterilize and prevent exposing relevant parts of the system to contaminants.

It is another objective of the apparatus to provide for the means and method to make a sterile or aseptic connection that may be simple to make, may be simple to use, may use what may be less complex connectors, may use what may be modified connectors, may use what may be added parts to existing connectors and may be able to easily replace parts as needed.

A means and method embodying the invention are hereinafter described, by way of example, referenced to the accompanying drawings.

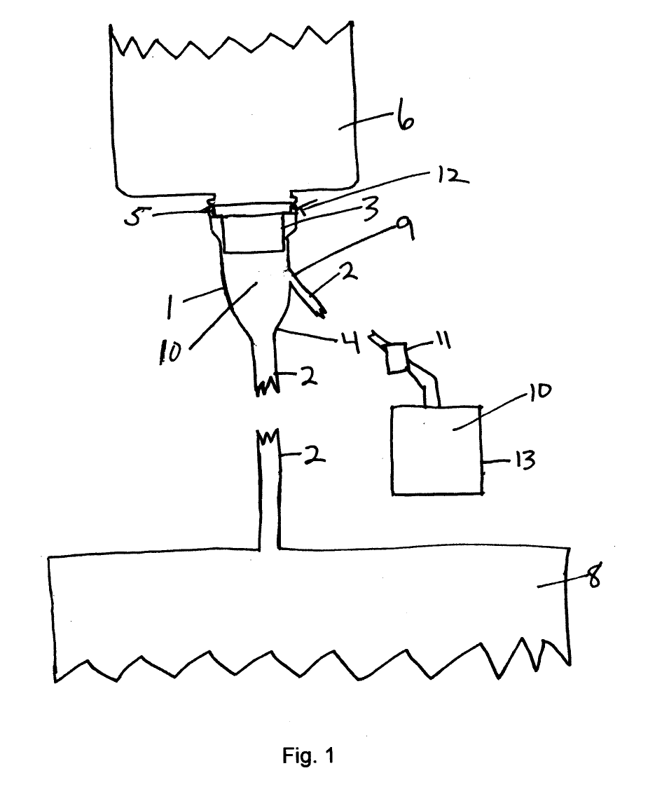

FIG. 1 shows a view providing substantially at least the elements from embodiment 1

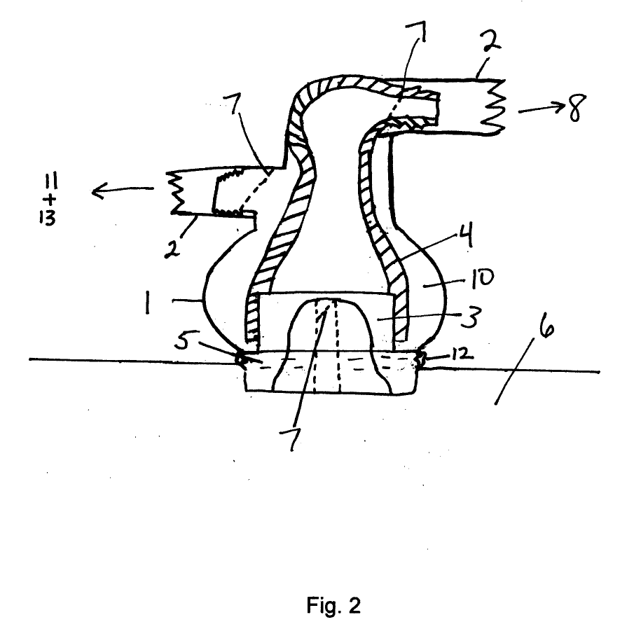

FIG. 2 shows a view providing substantially at least the elements from embodiment 2

### DETAILED DESCRIPTION

While this invention includes embodiments in different forms, the embodiments shown in the drawings and described herein are to be considered exemplifications of the principles of the invention and are not intended to limit the broad aspect of the invention to the embodiments illustrated and described herein.

### First Embodiment

A means and method FIG. 1 that facilitates the process of making an aseptic connection between material container 6 and terminus 8 or other elements that need an aseptic connection of hoses or tubes 2 by interspersed connectors. Enclosure 1 may be attached to or part of the terminus side connector 4. Enclosure 1 may have a foramen of a predetermined size at the container 6 end that will fit over all or a portion of material side connector 3 and may be of a predetermined internal diameter so that it may allow the completion of joining of connectors 3 and 4. The opposite foramen may easily fit over all or parts of terminus side connector 4 and over or within enclosure attachment 5. Enclosure attachment 5, may maintain enclosure 1 in place surrounding some or all of connectors 3 and 4 and may use a securing means 12. The apparatus remains in place and may prevent contamination of a system during the connection phase through to depletion of material container 6. It creates an entirely enclosed aseptic or sterile field to make the connection within and subsequently is used to sterilize the internal and external portions of connectors 3 and 4 and maintain sterility of the connectors 3 and 4 and may sterilize the internal portions tubes or hoses 2 and any means of conducting into and through terminus 8 to the point it exits the terminus. Connectors 3 and 4 is such that it is discretionary which connector is male and which connector is female. In this embodiment, material side connector 3 is male and the terminus side connector 4 is female and enclosure 1 incorporates alternative sterilant connector 9 to facilitate the dispersal of sterilant 10 into enclosure 1.

To operate, enclosure 1 may be part of or may be pre-attached to the terminus side connector 4. While terminus side connector 4 and enclosure 1 is elevated with the foramen that will fit over the material side connector 3 being the elevated portion, sterilant 10 is dispersed gradually into enclosure 1 by means of alternative sterilant connector 9 through attached hose or tube 2 leading from sterilant source 13 propelled by sterilant propulsion mechanism 11. This begins the internal sterilization of terminus side connector 4 and the parts within enclosure 1 before continuing the connection process. When sterilant 10 reaches the unattached foramen of enclosure 1, sterilant propulsion mechanism 11 is halted and enclosure 1 is attached to material side connector 3 using enclosure attachment 5 and that may use securing means 12. This begins to allow for the sterilization of material side connector 3 and other exposed areas within enclosure 1. After a pre-determined time, sterilant propulsion mechanism 11 is started, then a material propulsion mechanism is started. Sterilant 10 is drawn from enclosure 1 into an internal material conduit of the terminus side connector 4, then through the hose or tube 2 that leads to terminus 8 and may continue through to the point it leaves the terminus by using a propulsion mechanism and dispersing mechanism for terminus 8. After a predetermined amount of time the various propulsion mechanisms are turned off. Sterilant 10 has sterilized the internal portions of Terminus side connector 4, internal portions of hose or tube 2, then sterilized internal portions of a conduit through terminus 8 and possibly through it to the point a material exits terminus 8. Once a predetermined amount of time has been completed for the completion of the sterilization process, connectors 3 and 4 are then brought towards each other decreasing the length of enclosure 1 until a connection is completed within enclosure 1. It is maintained in place as a completely enclosed system that has been sterilized and thus prevents further exposure to contaminates. Sterilant propulsion mechanism 11 may be but not limited to a peristaltic pump, reciprocating pump or other types that would prevent contact between the fluid and pump. It may have variable speeds.

Sterilant connectors may contain check valves 7 to prevent backflow from enclosure 1 into hose or tube 2 leading to sterilant source 13. Connectors 3 and 4 may also contain check valves 7 to prevent backflow into other portions of the system such as but not limited to the material container 6.

Securing means 12 may be a plurality of types and may include but not limited to screw on, clamp, wire clamp, banjo, slip joint nut with slip joint washer and compression clamp.

### Second Embodiment

A means and method FIG. 2 that facilitates the process of making an aseptic connection between material container 6 and terminus 8 or other elements that need an aseptic connection of hoses or tubes 2 by interspersed connectors. Enclosure 1 may be attached to or part of the terminus side connector 4. Enclosure 1 may have a foramen of a predetermined size at the container 6 end that will fit over all or part of material side connector 3 and may be of a predetermined internal diameter so that it may allow the completion of joining of connectors 3 and 4. The opposite foramen may easily fit over all or parts of terminus side connector 4 and over or within enclosure attachment 5. Enclosure attachment 5, may maintain enclosure 1 in place surrounding some or all of connectors 3 and 4 and may use a securing means 12. The apparatus remains in place and may prevent contamination of a system during the connection phase through to depletion of material container 6. It creates an entirely enclosed aseptic or sterile field to make the connection within and subsequently is used to sterilize the internal and external portions of connectors 3 and 4 and maintain sterility of the connectors 3 and 4 and may sterilize the internal portions tubes or hoses 2 and any means of conducting into and through terminus 8 to the point it exits the terminus. Connectors 3 and 4 is such that it is discretionary which connector is male and which connector is female. In this embodiment, material side connector 3 is male and the terminus side connector 4 is female and enclosure 1 may be directly connected by one of two hoses or tube 2 attached to terminus side connector 4 that may lead to sterilant source 13 to facilitate the dispersal of sterilant 10 into enclosure 1.

In this embodiment enclosure 1 is part of terminus side connector 4 and may have two hoses or tubes connected by an additional means of attachment, one for sterilant dispersal and the other for material transmission. A conduit of transmittal for sterilant might run parallel to a material transmission conduit within terminus side connector 4. Enclosure 1 that surrounds and may be a part of terminus side connector 4 or may be attached to it by means of enclosure attachment 5.

To operate enclosure 1 with terminus side connector 4 are attached to material side connector 3 using enclosure attachment 5 and may use securing means 12. A sterilant propulsion mechanism 11 is initiated to propel sterilant 10 from sterilant source 13 through hose or tube 2 that is connected to terminus side connector 4 and then through a means of transmittal which is then dispersed into enclosure 1. When sterilant 10 completely fills enclosure 1 sterilant propulsion mechanism 11 is stopped. After a predetermined amount of time sterilant 10 has sterilized all within enclosure 1. Sterilant propulsion mechanism 11 is started, then a material propulsion mechanism and what may be a dispersing mechanism of terminus 8 are started. Sterilant 10 is drawn from enclosure 1 into a material conduit in terminus side connector 4, then through the hose or tube 2 that leads to terminus 8 possibly through it to the point a material exits the terminus 8. After a predetermined amount of time sterilant propulsion mechanism 11, a material propulsion mechanism, and a dispersing mechanism are turned off. Sterilant 10 has then sterilized the internal portions, including what might be a material conduit of terminus side connector 4, tubes or hoses 2, to terminus 8 and as well as possibly through what may be a conduit to the point a material exits the terminus 8. At this point, connectors 3 and 4 are brought together by possibly using the portion of terminus side connector 4 that remains outside of enclosure 1 to bring them together until the connection within enclosure 1 is completed. A means of securing the internal connection may be used. It is maintained in place as a completely enclosed system that has been sterilized to prevent exposure to contaminates. Enclosure 1 and terminus side connector 4 may be of types that can be replaced as needed.

Seals such as but not limited to gaskets or O rings may be employed on connector 3 and 4 to create a seal for prevention of sterilant or material escaping and introduction of organisms into enclosure 1 and other parts of the system, as well as for creating a tighter fit for connector 3 and 4 at points of contact to perform a function that might assist in securing the connection.

An alternative sterilant connector 9 on enclosure 1 may be utilized, as well as other means such as but not limited to a bayonet connector to facilitate the dispersing of the sterilant 10 into enclosure 1.

Sterilant propulsion mechanism 11 may be a but not limited to a peristaltic pump, reciprocating pump, gravity, pistons, bellows, pressure pumps, or other types that would prevent contact between the fluid and pump. It may have variable speeds.

Sterilant connectors may contain check valves 7 to prevent backflow from enclosure 1 into hose or tube 2 leading to sterilant source 13. Connectors 3 and 4 may also contain check valves 7 to prevent backflow into other portions of the system such as but not limited to the material container 6.

Securing means 12 may be but not limited to a plurality of types and may be but not limited to include screw on, clamp, wire clamp, banjo, slip joint nut with slip joint washer, lever lock and compression clamp.

### Brief SUmmary of the Invention

[symbol]

What is claimed:

1. A system comprised of:
An enclosure that may have a predetermined longitudinal shape, cross-sectional shape and length and may have foramens at either end or both of a predetermined diameter so it may easily fit over connectors and may be over or within the enclosure attachment and it's dimensions and construction may facilitate joining of connectors within said enclosure and is such that said enclosure diameter may not interfere with the function of slidably in order that it may reduce distance between connectors until completion of the joining of said connectors.
Enclosure may be constructed of a food grade material and may be pliable to facilitate the joining of said connectors.
Enclosure may have a connection for hose or tube for sterilant disbursement from sterilant source into enclosure in order to make all that is contained within and what it may make contact with sterile or aseptic and may contain what may be a check valve.
Connectors may be but not limited to modified versions of connectors that may have enclosure attachments added to design so it may maintain enclosure in place or may have enclosure as a permanent part of connector or may form an enclosure over elements of the connector used to make the connection and may be discretionary as to the gender of the connector used on material connector and may be discretionary as to the gender of the connector used on terminus connector as long as there is one of a gender for a connector and the opposite gender for the other connector and may contain what may be a check valve as well as what might be seals such as but not limited to gaskets or O rings to create a more secure and enclosed environment.
Connector may have a connection for hose or tube from sterilant source into enclosure and may contain a conduit that is connected to tubes or hoses from sterilant source for sterilant disbursement into enclosure in order to make all that is contained within and what it may make contact with sterile or aseptic and may contain what may be a check valve.
Connector may utilize other means of connecting hoses or tubes such as but not limited to bayonet connectors.
A sterilant that may sterilize the portion of said connectors within said enclosure, their internal portions and the internal portion of the hose that leads from connector to a terminus and possibly some or all of parts within terminus through to the point it exits the terminus.
Sterilant may be but not limited to a hydrogen peroxide solution, electrolyzed water, electrolyzed water in combination with other constituents or combinations of food grade sterilants.
A propelling mechanism that may propel material and may propel sterilant by means of hose or tube may be incorporated and may be selected from a group consisting of but not limited to peristaltic pumps, gravity, pistons, bellows, and pressure.

2. The system recited in claim 1 may be comprised of an improvement by providing a means of said enclosure so it may keep all parts within said enclosure sterile and isolated for the entirety of the operation of pre-connecting through to disconnect.

3. The system recited in claim 1 may be an improvement providing the system uses only a sterilant to make the process of connecting aseptic.

4. The system recited in claim 1 may be an improvement providing a person with ordinary skills in relevant art may be able to operate and maintain the system.

5. The system recited in claim 1 may be an improvement providing attachment of enclosure to connectors may be operated manually.

6. The system recited in claim 1 may be comprised of an improvement by providing a means of enclosure being replaceable and may be located in an accessible area that may be outside of any apparatus as an external part of the unit that may have a need for the system in order that it may facilitate the replacement of enclosure and connectors.
